(19) United States
(12) Patent Application Publication
(10) Pub. No.: US 2004/0034093 A1
(43) Pub. Date: Feb. 19, 2004

(54) Methods for enhancing motor performance and/or endurance

(76) Inventors: Kenneth L. Hensley, Oklahoma City, OK (US); Robert A. Floyd, Oklahoma City, OK (US)

Correspondence Address: FULBRIGHT & JAWORSKI L.L.P., 600 CONGRESS AVE., SUITE 2400, AUSTIN, TX, 78701, US

(21) Appl. No.: 10/438,179

(22) Filed: May 14, 2003

Related U.S. Application Data
(60) Provisional application No. 60/384,270, filed on May 30, 2002.

(51) Int. Cl. A61K 31/355

(52) U.S. Cl. 514/458

(57) ABSTRACT

The present invention provides methods for enhancing muscle performance and/or endurance by administering a composition comprising a desmethyl tocopherol. Athletic performance and sports medicine in general are benefited by the methods of the invention. In addition, the invention provides therapeutic utility to muscle disorders that involve nitrative stress and/or inflammation.

The present application claims the priority of U.S. Provisional Application Serial No. 60/384,270, filed May 30, 2002. The entire contents of the above-referenced application is incorporated herein by reference and without disclaimer.

The government owns rights in the present invention pursuant to grant number AG18945-01 from the National Institutes of Health (NIA).

### BACKGROUND OF THE INVENTION

1. Field of the Invention

The present invention relates generally to the fields of antioxidant therapies and muscle physiology. More particularly, it concerns methods to enhance motor performance and muscle endurance with compositions comprising desmethyl tocopherols, such as but not restricted to gamma tocopherol (γT).

2. Description of Related Art

Vitamin E, also known as alpha-tocopherol (αT), belongs to a class of molecules called the tocopherols, and is well known for its antioxidant properties. As αT is the major tocopherol in the body most of the ongoing research regarding antioxidant properties is focused primarily on this molecule. However, several other tocopherols, such as desmethyl tocopherols also exist, one example being γ-tocopherol (γT). γT is a natural product of plant origin and human plasma γT concentration is variously reported as between 5-30% that of αT (Handelman et al., 1985).

The ability to scavenge free radicals generated by reactive nitrogen species (RNS), which reduces/inhibits nitrative stress, is one of the desired property of an antioxidant. With respect to scavenging RNS, αT appears to be only marginally effective in comparison to γT. For example, it is reported that the in vitro reaction of γT with NO₂ gas is 6 times more rapid than the corresponding reaction of αT (Cooney et al., 1993). Furthermore, exposure of αT (but not γT) to NO₂ caused the formation of a secondary nitrating species which can nitrate the target compound morpholine (Cooney et al., 1993). Cooney et al. also showed that γT was 4-fold more effective than αT at inhibiting neoplastic transformation of methylcholanthrene-treated C3H/10T1/2 fibroblasts, a process believed to involve nitrative stress (Cooney et al., 1993).

In another study, where liposomal αT or γT were exposed to synthetic peroxynitrite (ONOO⁻), γT was found to be twice as effective as αT at inhibiting lipid hydroperoxide formation (Christen et al., 1997). Moreover, the γT nitration rates were not influenced by the presence of αT. This latter finding suggests that nitration of γT may occur preferentially to the reaction with αT when both tocopherols are simultaneously exposed to a nitrating species. However, in a conflicting study, Goss et al., report that αT does spare γT in liposomes exposed to the superoxide and other NO— generating compounds (Goss et al., 1999).

Using an in vivo model of cardiovascular stress, the efficacy of αT and γT in reducing free radical damage was compared in tocopherol-depleted rats which were fed with either αT or γT for two weeks after chronic exposure to iron-dextran, an inducer of oxyradical stress (Dillard et al., 1983). While both αT and γT inhibited systemic lipid oxidation in the animals, γT was approximately 35% as effective as αT (Dillard et al., 1983). However, lipid nitration was not analyzed in this study. In yet another study, γT was shown to be 20-30 times more potent than αT at inhibition of iron-induced lipid peroxidation and occlusive thrombus formation (Saldeen et al., October 1999).

In U.S. Pat. No. 6,346,544, the present inventors showed that desmethyl tocopherols, such as γT, protect cardiovascular tissues from nitrative stress. Animal models were used to demonstrate that γT scavenges reactive nitrogen species (RNS) during thrombosis in vivo; that γT protects the mitochondrial enzyme α-KGDH against nitrative stress; and that γT scavenges RNS in smokers and hypertensive individuals.

Protein nitration due to RNS is also known to occur in human neurodegenerative disease such as Alzheimer's, amyotrophic lateral sclerosis (ALS), and Parkinson's disease (Hensley et al., 1998; Smith et al. 1997; Tohgi et al., 1999; Liberatore et al., 1999). However, only αT has been evaluated for its ability to slow the progression of Alzheimer's disease in one large-scale clinical trial (Sano et al., 1997) and future trials using αT are planned (Grundman, 2000). In a co-pending U.S. patent application Ser. No. 09/794,293, incorporated herein by reference, the present inventors have shown that desmethyl tocopherols can reduce and prevent neurodegeneration.

### SUMMARY OF THE INVENTION

The present invention demonstrates that desmethyl tocopherols are superior to α-tocopherol (vitamin E) with respect to enhancing motor performance and/or muscle endurance in individuals.

Disclosed is the use of desmethyl tocopherols such as γ-tocopherol as scavengers of reactive nitrogen species in muscle tissues that are exerted or exposed to an inflammatory stress. Such reactions are seen in muscle tissues during intense muscle activity, such as exercise during athletic training. Such reactions also occur during muscle diseases such as muscle injuries during athletic activities, muscular dystrophies, neuromuscular diseases, myasthenia gravis, multiple sclerosis, amyotrophic lateral sclerosis, age-related sarcopenia and the like. Thus, the invention provides advantages to the general areas of sports medicine and athletic performance in addition to the treatment of myopathies.

Therefore, provided are methods of enhancing motor performance and/or muscle endurance comprising administering to a subject a composition comprising an effective amount of at least one desmethyl tocopherol. “Effective amount” is defined here as an amount of a desmethyl tocopherol that can enhance, improve, or boost muscle endurance and/or muscle performance; and/or the amount of a desmethyl tocopherol that can reduce, decrease, inhibit or abrogate muscle tissue damage due to intensive exercise/training; and/or the amount of a desmethyl tocopherol that can reduce, decrease, inhibit or abrogate excessive free radical production in muscle tissues.

In some embodiments, the desmethyl tocopherol has the general structure:

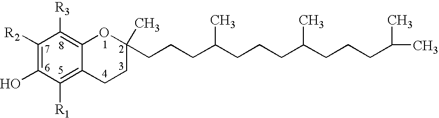

or is an isomer of such a structure,

wherein, at least one of the set R₁, R₂ and R₃ is a H atom, and R₁ may be —H, —CH₃, —CH₂CH₃, —OH, —O—Y, where Y is an alkyl moiety, a halogen, or a —NO₂; R₂ may be —H, —CH₃, —CH₂CH₃, —OH, —O—Y, where Y is an alkyl moiety, a halogen, or a —NO₂; and R₃ may be —H, —CH₃, —CH₂CH₃, —OH, —O—Y, where Y is an alkyl moiety, a halogen, or a —NO₂. Pharmaceutical formulations are also contemplated.

Additionally, the alkyl tail of the molecule may be comprised of either saturated or unsaturated variants, where unsaturated variants comprise the chemical subclass of tocotrienol tocopherols. Thus, the use of tocotrienol tocopherols is also contemplated in this invention. Since the main bioactive function of the above structure is the phenolic head group, any stereoisomer of the tocopherol may be used. Furthermore, since the main bioactive function of the above structure is the phenolic head group, any carbon can be eliminated from the carbon centers labeled 2-4 in the structure above. Furthermore, the —OH group can be esterified or otherwise modified to form a prodrug or a more water-soluble derivative such as an ester, which would regenerate the —OH group in vivo. These and other homologs of the tocopherols can be chemically synthesized or isolated from natural products by methods that are known to the skilled artisan.

In some aspects, the desmethly tocopherols localize to lipid environments and scavenge reactive nitrogen species (RNS) by virtue of a phenolic structural element lacking one or more methyl substituents on the phenolic ring system. The capability to differentially partition and/or scavenge RNS imparts superior effectiveness of desmethyl tocopherols as motor/muscle performance enhancing agents. Derivatives of γ-tocopherol (or other desmethyl tocopherols) which retain the structure of a phenolic ring lacking a H atom near the —OH group would also be useful as protectant against nitrative stress in muscular conditions.

In some embodiments of the invention, the desmethyl tocopherol is gamma tocopherol. Other non-limiting examples of desmethyl tocopherols include the desmethyl tocotrienols such as, desmethyl tocotrienol [3,4-dihydro-2-methyl-2-(4,8,12-trimethyltrideca-3′(E),7′(E), 11′-trienyl)-2H-1-benzopyran-6-ol]; didesmethy tocotrienol [3,4-dihydro-2-(4,8,12-trimethyltrideca-3′(E),7′(E), 11′-trienyl)-2H-1-benzopyran-6-ol]; as well as other desmethyl tocopherols such as, β-tocopherols; δ-tocopherols or tocol.

In some aspects, the desmethyl tocopherol may be comprised in a mixture of other tocopherols. In yet other aspects, the desmethyl tocopherol is a mixture of isomers.

The desmethyl tocopherol component may be isolated from natural sources. Examples of natural sources that are rich in desmethyl tocopherols such as γT, include soy beans, nuts, monounsaturated vegetable oils, whole grains, and wheat germ. Alternatively, the desmethyl tocopherols may be synthesized chemically or biochemically by methods well known in the art.

In some embodiments, since the desmethyl tocopherols are intended for use in pharmaceutical compositions, they may be provided in a substantially pure form. By “substantially pure” it is meant that a composition comprising the desmethyl tocopherol may comprise at least 50% desmethyl tocopherol, more suitably at least 75% desmethyl tocopherol and preferably at least about 95% desmethyl tocopherol and even more preferably at least about 98-99% or more of the desmethyl tocopherol.

In other embodiments, the desmethyl tocopherol is a water-soluble ester. In yet other embodiments, the desmethyl tocopherol is administered as a prodrug.

In some embodiments, the composition further comprises another agent. Such an agent may be an antioxidant such as α-tocopherol, β-carotene, vitamin B12, folic acid. Alternatively, the agent may be a drug such as an anti-inflammatory agent.

In the methods of the present invention, the tocopherols are administered in a safe and effective amount enhance muscle performance. In some embodiments, the dosage of desmethyl tocopherol administered is from about 100 mg/day to about 1000 mg/day. Thus it is contemplated that one may use, ranges from about 100 mg/day, 150 mg/day, 200 mg/day, 250 mg/day, 300 mg/day, 350 mg/day, 400 mg/day, 450 mg/day, 500 mg/day, 550 mg/day, 600 mg/day, 650 mg/day, 700 mg/day, 750 mg/day, 800 mg/day, 850 mg/day, 900 mg/day, 950 mg/day to about 1000 mg/day. Intermediate ranges are also contemplated, for example one may use 110 mg/day, or 270 mg/day, or 365 mg/day or 576 mg/day and so on. It will be understood that the exact method of administration and dosages of administration will be decided and adjusted at the time of administration, depending on the individual needs of a subject, taking into consideration factors such as, age, disease, gender, performance status, etc., and such adjustments will be made by a trained physician. Therefore, the invention is in no way limited by the doses set forth.

Several routes for administration of the composition are contemplated and these include oral, intramuscular, parenteral, or intrathecal.

Also provided by the invention are methods for reducing or inhibiting muscle tissue damage resulting from reactive nitrogen species comprising administration to a subject a composition comprising an effective amount of at least one desmethyl tocopherol.

Further provided are methods of preserving mitochondrial function in muscle tissue exposed to nitrative stress comprising the administration to a subject a composition comprising an effective amount of at least one desmethyl tocopherol.

The invention also provides methods of treating or preventing a condition wherein motor performance and/or muscle endurance is compensated comprising administering to a subject a composition comprising an effective amount of at least one desmethyl tocopherol. Examples of such condition include muscular dystrophies, muscle injuries, neuromuscular disorders such as myasthenia gravis, multiple sclerosis, amyotrophic lateral sclerosis, age-related sarcopenia and the like.

In the therapeutic methods of the present invention, the tocopherols are administered in a safe and effective amount to scavenge reactive nitrogen species and slow the progression of nitrative stress in muscle tissue that are exerted or are afflicted by a disorder. Furthermore, in the therapeutic methods an “effective amount” is defined as an amount of the desmethyl tocopherol that can reduce, decrease, inhibit or abrogate muscle tissue damage due to reactive nitrogen species (RNS), preserve mitochondrial function in muscle tissue exposed to RNS; and/or improve, prevent or rectify motor performance and/or muscle endurance in physiological conditions where such functions are compensated.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A & 1B. FIG. 1A. Depicts tocopherol structures. Arrows indicate the 5 position of the chromanol ring system, which is methylated in α-tocopherol (vitamin E) but not in γ-tocopherol. This structure difference allows γ-tocopherol to scavenge RNS in a manner that α-tocopherol cannot. The product of the scavenging reaction is 5-nitro-γ-tocopherol. FIG. 1B. Shows pathways for generation of nitrating agents and their subsequent reaction with phenolic substrates such as tyrosine or γT.

FIG. 2. Comparison of muscle endurance and/or motor performance in mice fed with α-tocopherol (vitamin E), γ-tocopherol, and control mice fed on a basal diet lacking tocopherols.

FIG. 3. Survivability curves for G93A-SOD1 mice fed with α-tocopherol (vitamin E), γ-tocopherol, and control mice fed on a basal diet lacking tocopherols.

### DESCRIPTION OF ILLUSTRATIVE EMBODIMENTS

The present invention demonstrates the superiority of desmethyl tocopherols, such as γ-tocopherol, as protectors against nitrative damage in muscle tissue. The results described here are novel in several respects. Particularly, the results demonstrate that gamma tocopherol (also referred to in this specification as γ-tocopherol or γT) is superior to α-tocopherol (i.e., vitamin E, a fully alkylated tocopherol) in systems where nitrative stress is a relevant phenomenon. The invention of this utility for desmethyl tocopherols is not obvious to most skilled practitioners of the art of antioxidant therapy. This contention is demonstrated by the fact that α-tocopherol is the only tocopherol that is being currently advocated and used as an antioxidant supplement to improve muscle performance in athletes and in the treatment of muscle fatigue. Moreover, efforts are underway to create crop plants overproducing α-tocopherol when it is known that oral supplementation of humans with α-tocopherol actually depletes the human body of γ-tocopherol (Handelman et al., 1985; and personal observations).

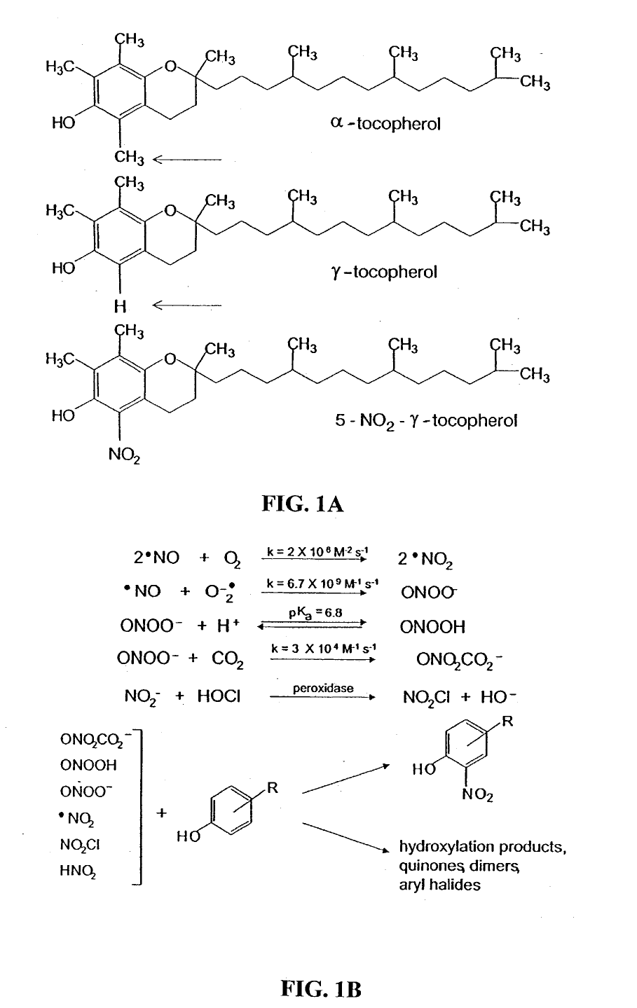

Recently, it has become appreciated that reactive nitrogen species (RNS) are significant in muscle fatigue and in many neuromuscular and muscular diseases. Such reactions also occur in muscle tissue during the stress of athletic exercise/training/competition. RNS are derived from the enzymatic oxidation of arginine via the intermediate nitric oxide free radical. Unlike oxygen-centered free radicals, reactive nitrogen species are not scavenged effectively by α-tocopherol. On the other hand, the present inventors have shown that γ-tocopherol can react easily with RNS. This is because of the presence of an open space on the chromanol head of the molecule (FIG. 1). The major product of γ-tocopherol reaction with RNS is 5-nitro-γ-tocopherol (5NγT, FIG. 1). The present inventors have previously shown that (A) γT protects the cardiovascular tissues from RNS much more effectively than αT; (B) γT is extensively nitrated in the brain of Alzheimer's disease patients; and (C) γT inhibits RNS toxicity to a critical mitochondrial enzyme α-ketoglutarate dehydrogenase, or αKGDH) which limits mitochondrial energy production. Thus, γT possesses unique biochemical functions from αT. In the present invention, γT has been shown to be a superior dietary supplement to maximize muscle energetics and performance, and extend athletic endurance. Thus, the present invention provides methods for enhancing muscle performance and endurance by administering compositions comprising desmethyl tocopherols. Furthermore, the therapeutic utility of desmethyl tocopherols in treating or preventing conditions where motor performance and/or muscle endurance are reduced or compensated is provided. This includes conditions such as, muscle injuries, muscular dystrophies, neuromuscular diseases such as myasthenia gravis, multiple sclerosis, amyotrophic lateral sclerosis, age-related sarcopenia and the like.

A. Tocopherols and Desmethyl Tocopherols

Tocopherols are a class of lipophilic, phenolic compounds of plant origin. The major tocopherol found in mammalian tissue is alpha tocopherol (α-tocopherol, αT or vitamin E; FIG. 1A), although significant quantities of demethylated (desmethyl) forms (particularly γ-tocopherol; FIG. 1A) are also present. α-Tocopherol acts as a free radical scavenger (i.e., a chain-breaking antioxidant) when the phenoxylic head group encounters a free radical:

Toc-OH+.L→Toc-O.+LH Toc-OH=tocopherol L.=lipid radical

The phenoxyl radical Toc-O. is much more stable, and less reactive, than L.. The aromatic nature of the tocopherol ring system, combined with steric and electronic influences from the methyl substituents, stabilizes the tocopheroxyl radical and thereby ends the lipid peroxidation process. Eventually, Toc-O. is reduced back to Toc-OH by ascorbate acting in conjunction with NADPH reductase.

While α-tocopherol is the major tocopherol in the body, other tocopherols exist. The second principle tocopherol in the human body is γ-tocopherol (γT), which, like α-tocopherol, is made by plants and taken into the human diet with foodstuffs. The plasma γT/αT ratio varies markedly among individuals. The proportion of γT/αT may be as low as 0.2% and as high as 30%. While both αT and γT are absorbed equally well by the gut, γT is packaged into lipoproteins less effectively than αT (Traber et al., 1992). Hence, αT supplementation results in a decrease in the systemic γT concentration (Handleman et al., 1985; Traber et al., 1992).

The γ-tocopherol and other desmethyl tocopherols are present in natural foods (particularly soy and wheat) in small amounts and are generally regarded as safe for human subjects. The biological activity of desmethyl tocopherols is associated with the chromanol head group of the molecule. This is to distinguish the tocopherols from tocotrienols, which inhibit cholesterol biosynthesis the activity of which is resident in the unsaturated lipid tail of the tocotrienol molecule. Gamma tocopherol (and other desmethyl tocopherols) may be chemically synthesized or isolated from natural products and such methods are well known in the art. For example, general methods for synthesizing tocopherols from phytol derivatives; and methods for isolating the tocopherols from natural products, are reviewed in Vandamme (1992), incorporated herein by reference in its entirety.

The present invention provides that desmethyl tocopherols, such as γT, enhance motor performance and muscle endurance. This is due to their ability to scavenge reactive nitrogen species (RNS) more effectively than α-tocopherol.

Several desmethyl tocopherols are known in the art, and non-limiting examples include, γ-tocopherol, β-tocopherol, δ-tocopherol, tocol; and desmethyl tocotrienols such as, desmethyl tocotrienol [3,4-dihydro-2-methyl-2-(4,8,12-trimethyltrideca-3′(E),7′(E), 1′-trienyl)-2H-1-benzopyran-6-ol]; didesmethy tocotrienol [3, 4-dihydro-2-(4,8,12-trimethyltrideca-3′(E),7′(E), 111′-trienyl)-2H-1-benzopyran-6-ol], and all these as well as their isomeric forms are contemplated to be useful in the methods of the invention.

It is also contemplated that one may use an analog or a biologically functionally equivalent of a desmethyl tocopherol for the methods of the invention. By rational drug design one can produce structural analogs of biologically active compounds. By creating such analogs, it is possible to fashion drugs which are more active or stable than the natural molecules. In one approach, one would obtain the three-dimensional structure for the desmethyl tocopherol and design molecules that have similar structures. This could be accomplished by X-ray crystallography, computer modeling or by a combination of both approaches. An alternative approach, involves the random replacement of functional groups throughout the desmethyl tocopherol, and the resulting affect on function determined.

B. Nitrative Stress in Muscle

In the arena of athlete muscle performance, it is desirable to create conditions that permit competition or training at higher levels of resistance for a prolonged period of time. However, acute and intense anaerobic use of skeletal muscles often results in impaired athletic performance, with losses in force and work output, and increased onset of muscle fatigue, soreness, and dysfunction. It is now recognized that even a single exhaustive exercise session, or for that matter any acute trauma to the body such as muscle injury, resistance or exhaustive muscle exercise, or elective surgery, is characterized by perturbed metabolism that affects muscle performance in both short and long term phases. Both muscle metabolic/enzymatic activity and gene expression are affected. For example, disruption of skeletal muscle nitrogen metabolism as well as depletion of sources of metabolic energy occur during extensive muscle activity. Amino acids, including branched-chain amino acids, are released from muscles followed by their deamination to elevate serum ammonia and local oxidation as muscle fuel sources, which augments metabolic acidosis. In addition, there is a decline in catalytic efficiency of muscle contraction events, as well as an alteration of enzymatic activities of nitrogen and energy metabolism. Further, protein catabolism is initiated where rate of protein synthesis is decreased coupled with an increase in the degradation of non-contractible protein. These metabolic processes are also accompanied by free radical generation which further damages muscle cells.

Reactive oxygen species (ROS) and reactive nitrogen species (RNS), exemplified by the parent radicals: superoxide anions (for ROS cascades), and nitric oxide (for RNS cascades); as well as other free radical ions such as, hydrogen peroxide, hydroxyl radicals, and peroxynitrite are generally identified with pathological states as mediators of cellular injury. It is well known that muscle tissues that are fatigued and overworked also have elevated levels of ROS and RNS (Novelli et al., 1990; Barclay et al., 1991; Russo et al., 1991, Novelli et al., 1991; Hasegawa et al., 1997; Clarkson 1995; Powers and Lennon 1999). Exhaustive exercise has been shown to generate free radicals the effects of which have been evidenced by increases in lipid peroxidation, glutathione oxidation, and oxidative protein damage in muscle cells (see, Reid, 2001; and Vina et al., 2001 for review articles on the subject). Detection of ROS and RNS production muscle cells can be performed by various techniques including electron spin resonance, fluorescent assays, cyotchrome c reduction, chemiluminescence, hydroxylation of salicylate, and assays to measure nitration of phenylalanine or arginine.

Recovery from fatigue during acute and extended exercise requires reversal of metabolic and non-metabolic fatiguing factors. Known factors that participate in human muscle fatigue, such as lactate, ammonia, hydrogen ion, etc., provide an incomplete and unsatisfactory explanation of the fatigue/recovery process, and it is likely that additional unknown agents participate (Baker et al., 1997; Bazzarre et al., 1992; Dohm et al., 1985; Edwards 1983; MacDougall et al., 1992; Walser 1987). Several studies have also analyzed the effects of nutritional supplements and herbal supplements in enhancing muscle performance.

While α-tocopherol can scavenge ROS, they are not very effective against RNS. In limited trials in humans, α-tocopherol did not extend muscle endurance (reviewed in Clarkson 1995; Powers and Lennon 1999). In general, supplementation with antioxidants can reliably decrease biochemical correlates of oxidative stress in humans but without an associated increase in performance (Clarkson 1995; Powers and Lennon 1999). Novelli et al., (1990), showed that some free radical trappers including α-tocopherol extended rodent endurance during a swimming test.

While α-tocopherol can scavenge ROS, they are not very effective against RNS. The present invention shows that the desmethyl tocopherols are highly effective against RNS. Thus, the present invention describes previously unknown and surprising properties of desmethyl tocopherols. These properties also distinguish other antioxidants from desmethyl tocopherols in terms of function. Hence, the present invention provides a safe and effective method for scavenging RNS from muscle tissue by the use of a natural dietary supplement.

Aside from muscle performance during endurance exercise, free radicals and oxidative stress parameters are affected in pathophysiological states. A substantial body of data now suggests that oxidative stress contributes to muscle wasting or atrophy in pathophysiological states (reviewed in Clarkson 1995; Powers and Lennon 1999). For example, with respect to muscular disorders where both muscle endurance and function are compensated, the role of nitric oxide (NO), has been implicated. In muscular dystrophies, especially those due to defects in proteins that make up the dystrophin-glycoprotein complex (DGC), the enzyme that synthesizes NO, nitric oxide synthase (NOS), has been associated. Recent studies of dystrophies related to DGC defects suggest that one mechanism of cellular injury is functional ischemia related to alterations in cellular NOS and disruption of a normal protective action of NO. This protective action is the prevention of local ischemia during contraction-induced increases in sympathetic vasoconstriction. Rando, (2001), has shown that oxidative injury precedes pathologic changes and that muscle cells with defects in the DGC have an increased susceptibility to oxidant challenges. Excessive lipid peroxidation due to free radicals has also been shown to be a factor in myopathic diseases such as McArdle's disease (Russo et al., 1997). Furthermore, mitochondrial dysfunction is a well-known correlate of age-related muscle wasting (sarcopenia) and free radical damage has been suggested, though poorly investigated, as a contributing factor (reviewed in Navarro et al., 2001). It is contemplated that the methods of the present invention will also be effective in the treatment of muscle related pathological conditions.

C. Pharmaceuticals

In practice, the desmethyl tocopherols such as γ-tocopherol will be formulated in a manner allowing safe delivery of effective amounts or doses to humans. “Effective amount” is defined as an amount of the agent that will enhance, improve, or boost muscle endurance and/or muscle performance; reduce, decrease, inhibit or abrogate muscle tissue damage due to reactive nitrogen species (RNS), preserve mitochondrial function in muscle tissue exposed to RNS; and/or improve, prevent or rectify motor performance and/or muscle endurance in physiological conditions where such functions are compensated.

The relative stability and lipophilicity of desmethyl tocopherols, such as γ-tocopherol, make these compounds amenable to delivery in numerous possible formulations. Desmethyl tocopherols can be absorbed orally by mammals and oral administration is contemplated. Thus, formulations of desmethyl tocopherols may be formulated in pills, tablets, capsules, troches, lozenges, syrups, and the like. Alternatively, one may administer the compositions comprising the desmethyl tocopherols via intramuscular or parenteral methods. The desmethyl tocopherols may also be administered topically to inflamed skin or gum/mouth tissue as a cream or gel, or can be inhaled as an aerosol.

Pharmaceutical compositions comprising effective amounts of desmethyl tocopherols may be dissolved or dispersed in a pharmaceutically acceptable carrier or medium to form therapeutic formulations that may then be administered according to methods of the invention.

The compositions of the present invention can be formulated in standard pharmaceutical carriers for administration to subjects or patients in need thereof. These include saline, phosphate buffered saline, and other aqueous carriers, and liposomes, polymeric microspheres and other controlled release delivery devices, as are well known in the art.

The phrases “pharmaceutically or pharmacologically acceptable” refer to molecular entities and compositions that do not produce an adverse, allergic or other untoward reaction when administered to an animal, or a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The active compounds may be formulated for parenteral administration, e.g., formulated for injection via the intravenous, intraarthricular, intrathecal, intramuscular, sub-cutaneous, intralesional, or even intraperitoneal routes. Typically, such compositions can be prepared as injectibles, either as liquid solutions or suspensions; solid forms suitable for using to prepare solutions or suspensions upon the addition of a liquid prior to injection can also be prepared; and the preparations can also be emulsified.

The pharmaceutical forms suitable for injectible use include sterile aqueous solutions or dispersions; formulations including sesame oil, peanut oil or aqueous propylene glycol; and sterile powders for the extemporaneous preparation of sterile injectible solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

Formulations of neutral or salt forms are also provided. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectible compositions can be brought about by the use in the compositions of agents delaying absorption, for example, aluminum monostearate and gelatin.

Sterile injectible solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectible solutions, the preferred methods of preparation are vacuum-drying and freeze-drying techniques which yield a powder of the active ingredient plus any additional desired ingredient from a previously sterile-filtered solution thereof.

The preparation of more, or highly, concentrated solutions for local injection also is contemplated. In this regard, the use of DMSO as solvent is preferred as this will result in extremely rapid penetration, delivering high concentrations of the active agents to a small area.

Upon formulation, solutions will be administered in a manner compatible with the dosage formulation and in such amount as is diagnostically or therapeutically effective. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. Local or regional administration, with respect to an inflamed muscle, also is contemplated. Finally, systemic administration may be performed. Delivery via syringe or catherization is also contemplated.

In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated or diagnosed. The person responsible for administration will, in any event, determine the appropriate dose for the individual subject.

### D. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Demonstration of Enhanced Motor Performance by Gamma Tocopherol Supplementation

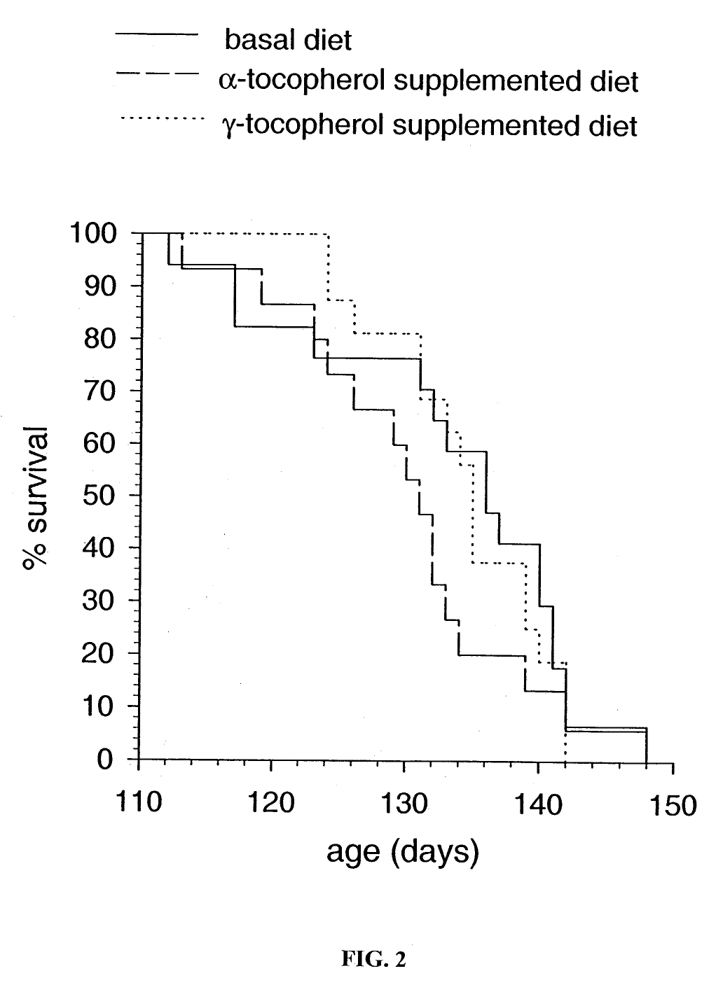

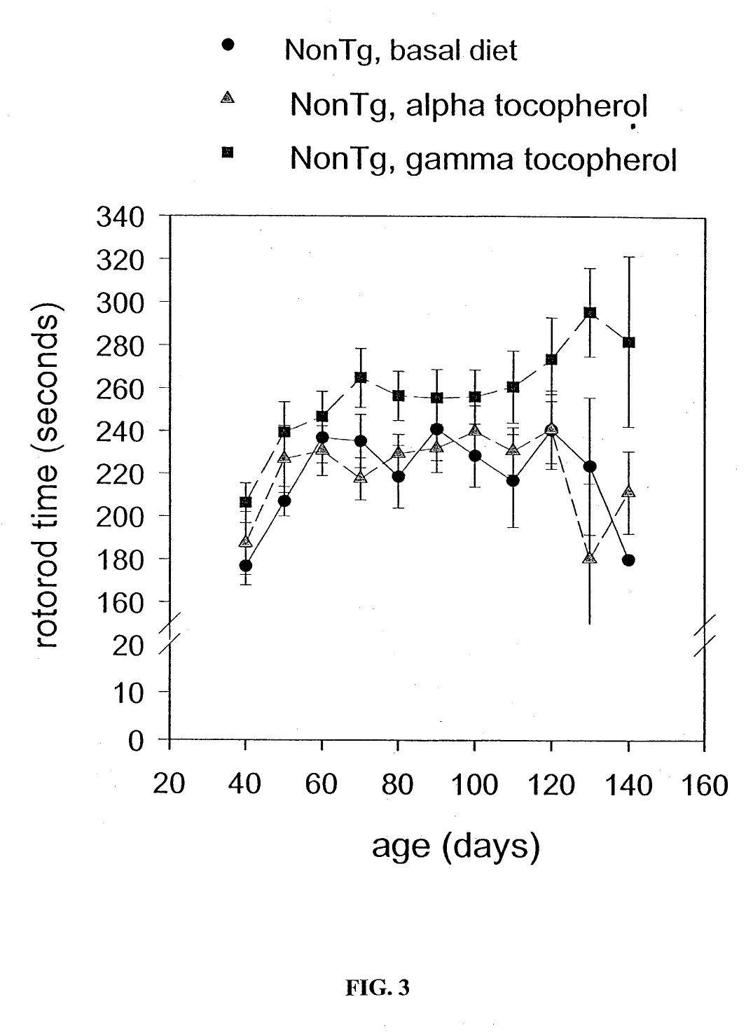

Normal, nontransgenic (NonTg) C57/B6 mice were studied for efficacy of γT on motor performance and/or enhanced muscle function. Beginning at 40 days of age, animals were fed a basal AIN93G laboratory animal diet containing 75 mg/kg α-tocopherol and negligible γ-tocopherol; or the same diet supplemented with an additional 200 mg/kg α-tocopherol or γ-tocopherol. Motor function was measured by a standard rotorod performance task (Klivenyi et al., 1999). Animals were placed on a motorized, rubber-coated metal rod that was set to rotate at 1 rpm increasing by 1 rpm every 10 seconds (Klivenyi et al., 1999). The speed of the rod rotation at which the mouse falls off is taken as a measure of motor competency (Klivenyi et al., 1999). Each mouse was tested in three trials every 10 days. As shown in FIG. 2 & FIG. 3, animals fed the γ-tocopherol supplement were significantly more capable of performing the motor function task than were animals fed either the basal diet or the α-tocopherol supplemented diet.

All of the compositions and/or methods disclosed and claimed herein can be made and executed without undue experimentation in light of the present disclosure. while the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 6,346,544

U.S. patent application Ser. No. 09/794,293

Baker et al., J. Appl. Physiol. 74:2294-2300, 1993.

Barclay and Hansel, Can J Physiol Pharmacol. 69(2):279-84, 1991.

Bazzarre et al., J Am. Coll. Nutr. 11:505-511, 1992.

Christen et al., Proc. Natl. Acad. Sci. USA 94:3217-3222; 1997.

Clarkson, P. M. Antioxidants and physical performance. Crit. Rev. Food Sci. Nutr. 35: 31-41; 1995.

Cooney et al., Proc. Natl. Acad. Sci. USA. 90:1771-1775, 1993.

Dillard et al., J. Nutr. 113:2266-2273; 1983.

Dohm et al., Fed. Proc. 44:348-352, 1985.

Edwards In: Biochemistry of Exercise, Proceedings of the Fifth International Symposium on the Biochemistry of Exercise (Kutrgen, Vogel, Poormans, eds.), 1983.

Goss et al., Arch. Biochem. Biophys. 363:333-340; 1999.

Grundman, Am. J Clin. Nutr. 630S-636S; 2000.

Handelman, et al., J. Nutr. 115:807-813; 1985.

Hasegawa et al, Free Radic Biol Med. 22(1-2):349-54, 1997.

Hensley et al., J. Neurosci. 18:8126-8132; 1998.

Klivenyi et al., Nature Med. 5:347-350; 1999 Liberatore et al, Nat Med 5:1403-1409; 1999.

MacDougall et al., Acta Physiol. Scand. 146:403-404, 1992.

Navarro, A.; Lopez-Cepero, J. M.; Sanchez del Pino, M. L. Front. Biosci. 6: D26-44; 2001.

Novelli et al., Free Radic Biol Med., 8(1):9-13, 1990.

Novelli et al., Pharmacol Res. 23(2):149-55, 1991.

Powers, S. K.; Lennon, S. L. Analysis of cellular responses to free radicals: Focus on exercise and skeletal muscle. Proc. Nutr. Soc. 58: 1025-1033; 1999.

Rando, Microsc Res Tech 55(4):223-35, 1001.

Reid, Med Sci Sports Exerc 33(3):371-6, 2001.

Remington's Pharmaceutical Sciences, 15^{th} ed., pages 1035-1038 and 1570-1580, Mack Publishing Company, Easton, Pa., 1980.

Russo et al., Med Hypotheses. 39(2):147-51, 1992.

Saldeen et al., J. Am. Coll. Cardiol. 34:1208-1215; 1999.

Sano et al., N Engl. J Med. 336:1216-1222; 1997.

Smith et al., J Neurosci 17:2653-2657; 1997.

Tohgi et al., Ann Neurol 46:129-131; 1999.

Traber, M. G.; Burton, G. W.; Hughes, L.; Ingold, K. U.; Hidaka, H.; Malloy, M.; Kane, J.; Hyams, J.; Kayden, H. J. Discrimination between forms of vitamin E by humans with and without genetic abnormalities of lipoprotein metabolism. J. Lipid Res. 33, 1171-1182; 1992.

Vandamme, E. J. Production of vitamins, coenzymes and related biochemicals by biotechnological processes. J Chem Technol Biotechnol 53: 313-327; 1992.

Vina et al., IUBMB Life 250(4-5):271-7, 2000.

Walser et al., Kidney Int. 32:123-128, 1987.

Williamson et al., Nitric Oxide, 6:221-227, 2002.

What is claimed is:

1. A method of enhancing motor performance and/or muscle endurance comprising administering to a subject a composition comprising an effective amount of at least one desmethyl tocopherol.

2. The method of claim 1, wherein the desmethyl tocopherol has the structure: 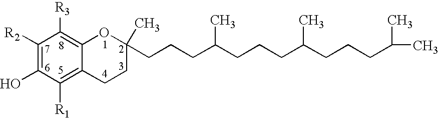
or an isomer thereof,
wherein, at least one of the set R₁, R₂ and R₃ is a H atom, and
R₁ is selected from —H, —CH₃, —CH₂CH₃, —OH, —O—Y, where Y is an alkyl moiety, a halogen, or a —NO₂;
R₂ is selected from —H, —CH₃, —CH₂CH₃, —OH, —O—Y, where Y is an alkyl moiety, a halogen, or a —NO₂; and
R₃ is selected from —H, —CH₃, —CH₂CH₃, —OH, —O—Y, where Y is an alkyl moiety, a halogen, or a —NO₂.

3. The method of claim 1, wherein said composition further comprises another agent.

4. The method of claim 2, wherein said agent is an antioxidant.

5. The method of claim 4, wherein the antioxidant is alpha tocopherol.

6. The method of claim 2, wherein said agent is a drug.

7. The method of claim 1, wherein the desmethyl tocopherol is a gamma tocopherol, a β-tocopherol, a δ-tocopherol, a tocotrienol, or a tocol.

8. The method of claim 1, wherein the desmethyl tocopherol is gamma tocopherol.

9. The method of claim 7, wherein the tocotrienol is desmethyl tocotrienol [3,4-dihydro-2-methyl-2-(4,8,12-trimethyltrideca-3′(E),7′(E), 11′-trienyl)-2H-1-benzopyran-6-ol]; or didesmethy tocotrienol [3,4-dihydro-2-(4,8,12-trimethyltrideca-3′(E),7′(E), 11′-trienyl)-2H-1-benzopyran-6-ol].

10. The method of claim 1, wherein the desmethyl tocopherol is comprised in a mixture of other tocopherols.

11. The method of claim 1, wherein the desmethyl tocopherol is a mixture of isomers.

12. The method of claim 1, wherein the desmethyl tocopherol is a water-soluble ester

13. The method of claim 1, wherein the desmethyl tocopherol is isolated from natural sources.

14. The method of claim 1, wherein the desmethyl tocopherol is synthesized chemically.

15. The method of claim 1, wherein the desmethyl tocopherol is substantially pure.

16. The method of claim 1, where the desmethyl tocopherol is administered as a prodrug.

17. The method of claim 1, wherein the amount of desmethyl tocopherol administered is from about 100 to about 1000 mg/day.

18. The method of claim 1, wherein the administration is oral, intramuscular, parenteral, or intrathecal.

19. A method for reducing or inhibiting muscle tissue damage resulting from reactive nitrogen species comprising administration to a subject a composition comprising an effective amount of at least one desmethyl tocopherol.

20. A method of preserving mitochondrial function in muscle tissue exposed to nitrative stress comprising the administration to a subject a composition comprising an effective amount of at least one desmethyl tocopherol.

21. A method of treating or preventing a condition wherein motor performance and/or muscle endurance is compensated comprising administering to a subject a composition comprising an effective amount of at least one desmethyl tocopherol.

22. The method of claim 21, wherein the condition is a muscle dystrophy, a neuromuscular disorder, McArdle's disease, myasthenia gravis, a muscle injury, multiple sclerosis, amyotrophic lateral sclerosis, age-related sarcopenia.

23. The method of claim 21, wherein the desmethyl tocopherol is a gamma tocopherol, a β-tocopherol, a δ-tocopherol, a tocotrienol, or a tocol.
